(19) United States
(12) Patent Application Publication
(10) Pub. No.: US 2025/0153163 A1
(43) Pub. Date: May 15, 2025

(54) PRE-MIX TEST VESSEL

(71) Applicant: ATOMO DIAGNOSTICS LIMITED, New South Wales (AU)

(72) Inventors: Rohit Ketkar, New South Wales (AU); Alan Keane, New South Wales (AU); Chandra Sukumar, New South Wales (AU); Mark Smith, New South Wales (AU); Toby Wheeler, New South Wales (AU); Huw Wallis, New South Wales (AU); Andrew Hung, New South Wales (AU); Jake Harris, New South Wales (AU); John Kelly, New South Wales (AU)

(21) Appl. No.: 18/427,350

(22) PCT Filed: Jul. 29, 2022
(86) PCT No.: PCT/AU2022/050812

§ 371 Date: Jan. 30, 2024

(30) Foreign Application Priority Data
Jul. 30, 2021 (AU) 2021902352
Feb. 11, 2022 (AU) 2022900285

(51) Int. Cl. B01L 3/00

(52) U.S. Cl. CPC B01L 3/5023; B01L 3/502715; B01L 3/5029; B01L 2200/027; B01L 2200/0621; B01L 2200/10; B01L 2200/16; B01L 2300/047; B01L 2300/069; B01L 2300/0825

(57) ABSTRACT

The present invention provides a pre-mix device for use with a test unit. The device includes a vessel, an integrated reservoir of test fluid, and a discharge port. A sample may be received into the vessel and the test fluid may be discharged from the reservoir into the vessel, so that a mixture of the sample and the test fluid may be released from the discharge port to a test unit.

### TECHNICAL FIELD

The present invention relates to test procedures carried out on user operated test devices, such as point of care and self-test devices, for example lateral flow or other rapid test devices.

### BACKGROUND OF THE INVENTION

Testing for various indications using single use, relatively inexpensive devices is a growing part of medical practice, as well as in other fields of activity. These may be, for example, lateral flow or other rapid test devices, intended for point of care, professional or at home testing. The tests may be for use with samples such as blood (including serum and plasma), saliva, mucus, urine or faeces. The test may be taken with a sample collected using, for example, a nasal or throat swab, or other suitable test protocols. Typical tests include those for specific infective agents or antibodies, metabolites, specific molecules, or combinations of these.

Self-testing using a nasal swab has become a common technique, particularly in the context of COVID testing. In a typical procedure, a swab is inserted into a nostril and then rotated. The procedure is repeated in the other nostril. The swab is then inserted into a tube, often pre-filled with a test fluid. The swab is agitated or rotated to disperse the sample into the test fluid, and the swab is removed. The top of the tube is then capped and used to dispense drops of mixed sample and test fluid into the sample port of a test unit. The test unit typically includes T (test) and C (control) lines, which are read visually by the user. In some cases, a UV light may be required to view the result.

Professional testing using a nasal swab based test and a reader is also widely practiced. In such procedures, after a similar series of sample acquisition steps, the test unit is placed into an automated reader. The reader determines the outcome of the test, for example using controlled illumination and optical sensing, and displays a result.

Saliva based testing has practical advantages over some existing technologies, for example nasopharyngeal swabs. It is less invasive for the person being tested, and more amenable to self-testing, or testing in a public facility or business, for example as a condition of entry or attending work.

In such testing, the collection of saliva in an easy way, which delivers the sample to the test device in a reliable way, premixed with a buffer or other reagent, is an important consideration. The saliva may contain active virus or other contagious material, and so the safe handling and management of the sample are important.

One approach to collecting saliva is to suck or orally impregnate a swab material, and store it in a container for later testing, for example as in the commercially available SalivaBio Oral Swab, from Salimetrics https://salimetrics.com/collection-method/oral-swab-saliva-collection-device/. However, this is not useful for rapid test or self-test situations.

Another approach is to provide a container into which the person being tested spits and a buffer or other extraction fluid is added to the saliva in the container from a separate dropper, and after mixing together, this mixture is then manually added to the test material. An example is https://bioservuk.com/product/rapid-responsetm-covid-19-saliva-rapid-antigen-test/. This approach requires a kit of components to be assembled, and is relatively complex for the user.

In all approaches in which a sample is disbursed into a test fluid and then a certain number of drops are placed into the sample port of the test device, only that part of the sample which is disbursed into the drops is presented to the test material.

Another method of testing a sample is disclosed in WO/2014/000037 by Axxin Pty Ltd. This patent discloses a nucleic acid amplification and detection kit or apparatus that constitutes a platform for collecting a nucleic acid sample, performing nucleic acid amplification on the sample, and performing lateral flow tests on the resulting amplification products.

It is an object of the present invention to facilitate pre-mixing of samples with a test fluid before carrying out a test procedure with a test device.

### SUMMARY OF THE INVENTION

In a broad form, the present invention provides a vessel having an opening to permit sample collection, an integrated test fluid reservoir to release test fluid into the vessel after it is closed, and a transfer port to allow at least part of the mixture of test fluid and sample to enter a test unit.

According to one aspect, the present invention provides pre-mix device for use with a test unit, including a vessel, an integrated reservoir of test fluid, and a discharge port, wherein a sample may be received into the vessel, the test fluid may be discharged from the reservoir into the vessel, so that a mixture of the sample and the test fluid may be released from the discharge port to a test unit.

According to another aspect, the present invention provides a medical test device, including a test unit including a body, a test material and a sample port to access the test material; and a pre-mix device, such that in use a sample is received into the pre-mix device, mixed with a test fluid, and then discharged into the sample port.

Implementations of the invention allow for a simple and reliable collection, mixing with test fluid and transfer of samples for use in a simple test unit, such as a rapid test. The present invention in suitable implementations can facilitate a simple and reliable pre-mixing vessel for receiving a sample and mixing with a test fluid such as a buffer, so that a sample of the mixture may be presented to a test unit. An advantage is that a more reliable, less complex sample collection and delivery system is thereby facilitated.

Implementations may further provide for a dry chemical reagent or test component, for example a lyophilised buffer, to be mixed in the vessel with the sample.

A further advantage of suitable implementations is that substantially all of the transferred sample is held in the vessel and mixed with the predetermined volume of test fluid, so that the concentration of sample in the mixed fluid dispensed to the test unit is optimised.

### BRIEF DESCRIPTION OF THE DRAWINGS

Illustrative embodiments of the present invention will be described in connection with the accompanying figures, in which:

FIG. 1 is a perspective view of a test unit adapted for use with the present invention;

FIGS. 2A and 2B are perspective views of a closed and open saliva vessel according to one implementation of the present invention;

FIG. 2C is a view in cross section of the vessel of FIG. 2A;

FIG. 3 illustrates the use of the vessel of FIG. 2 with a test unit;

FIG. 4 illustrates the stages of use for a second implementation;

FIG. 5 is a series of views of a mechanism for the second implementation;

FIG. 6 illustrates a third implementation;

FIG. 7 illustrates a fourth implementation;

FIG. 8 illustrates a fifth implementation;

FIG. 9 illustrates an alternative form of FIG. 4 for an alternative test unit; and

FIGS. 10A to 10C illustrates an alternative plus type release mechanism;

FIG. 11 illustrates the stages of use of another alternative form of FIG. 4;

FIG. 12 is a side view of a pre-mix unit in its resting condition;

FIG. 13 is a cross section view of the pre-mix unit after the test fluid is released;

FIG. 14 shows the vertical serration ribs;

FIG. 15 shows the funnel shaped ribs;

FIG. 16 shows the ribs extracting sample from the swab;

FIG. 17 shows the pre-mix unit placement on a test unit;

FIG. 18 shows the pre-mix fluid being delivered to the test unit; and

FIG. 19 shows a user directly adding drops of blood sample in the pre-mix unit.

### DETAILED DESCRIPTION

The present invention will be described with reference to several examples of a testing devices and pre-mix devices, using a lateral flow test. However, the general principle of the present invention is applicable to any tests for which a sample is required to be collected, mixed with a test fluid, and then tested. The present invention is not specific to any specific test type or system.

The present invention will be described primarily in the context of saliva and nasal swabs, but the invention may be applied to a variety of sample types, for example mucus, nasal or other swabs, blood, urine and faeces.

It will be understood that the present invention is concerned with the mechanical and fluid transport aspects of samples and test fluids for such a test device. Any suitable chemical, biochemical or other medical test may be used. The examples may be understood to include a lateral flow type test, for example for COVID-19 or similar respiratory viruses, with a conventional outcome of developed test and control lines. However, the test could operate electronically, be assessed automatically (for example by an optical system that analyses test and control lines), or operate in any other suitable way. The present invention may be utilised with any alternative kind of test, for example lab on a chip, molecular assays, loop mediated isothermal amplification, nucleic acid tests and other tests requiring a sample to be pre-mixed with a test fluid or dry material.

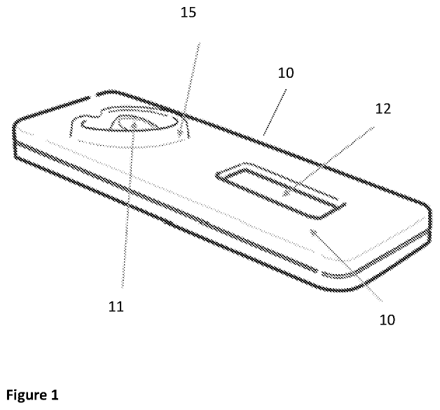

FIG. 1 illustrates a suitable test unit 10 for use in implementations of the present invention. It is largely conventional in set up, having a generally rectangular shape, with a test strip (not visible) positioned inside. A sample port 11 allows a sample and buffer to be deposited onto a section of the test strip. The resulting indication on the test strip is visible in the result window 12. Typically, a capillary process moves the sample through the test strip, which selectively binds components of the sample to chemical tag compounds, for example gold nanoparticles, latex beads, quantum dots, or cellulose nanobeads. If the test is positive, a test line appears in the window, as well as a control line which indicates a valid test has occurred. If the test is negative, no test line is produced. The test unit 10 is a single use device.

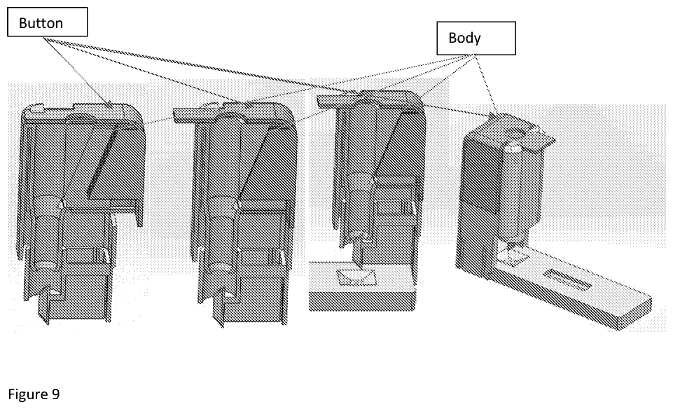

The test unit illustrated includes a raised rim 15 to assist in location of the pre-mix vessel, as will be explained further. However, this is not present in all test units, and the present invention can be employed using a test unit which has no such alignment features, for example as shown in FIG. 9.

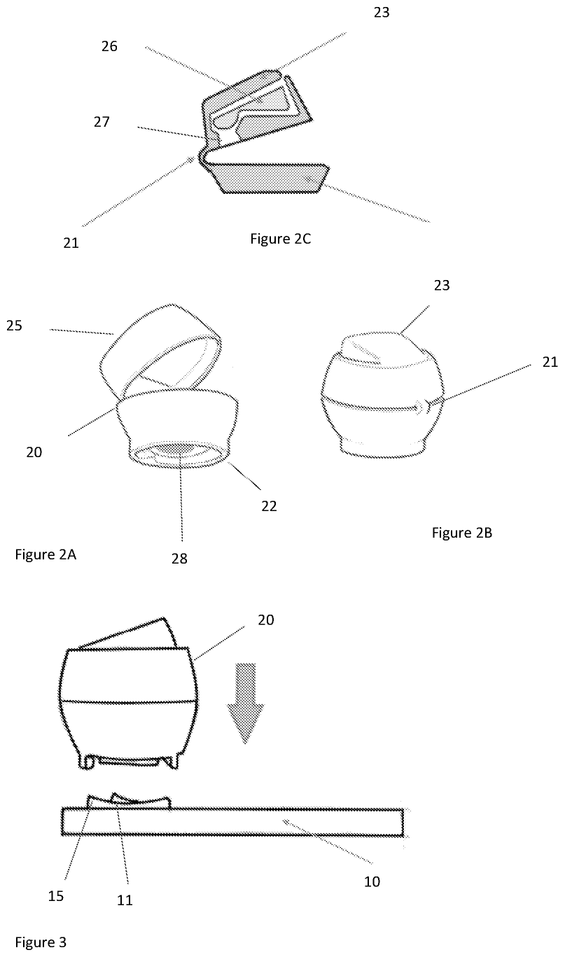

FIGS. 2A, 2B and 2C illustrate the pre-mix vessel 20. It consists of an upper section 25, which also acts as a cover, and a lower section 22 to receive the saliva sample, the upper section 25 and lower section 22 being joined by hinge 21. The vessel may be formed by any suitable means, for example, from injection moulded polymer.

The lower section 22 includes a discharge port 28 at the bottom, which is selectively controllable to allow delivery of the mixed sample and test fluid. In the illustrated form, the discharge port is an opening covered with a foil or similar layer, which is ruptured when vessel 10 is positioned on the test unit, as will be explained in more detail below. However, the discharge port could be operable in another way, for example with a removeable tab, a biased valve or flap that opens on contact, or a layer of absorbent or porous material such as glass fibres which allows for the flow to occur when in contact with the test strip or in any other suitable way.

In an alternative implementation, the discharge through the discharge port could be controlled by viscosity. One or more smaller holes could be provided in the discharge port, through which relatively viscous saliva cannot flow. However, once the test fluid is added, potentially including surfactants or other viscosity modifying additives, the viscosity lowers sufficiently that the mixture of saliva and test fluid will flow into the sample port. In this implementation, the vessel is preferably placed into position on the test unit prior to discharge of the test fluid.

The upper cover includes a button 23, which overlays a blister unit 26 filled with a test fluid. When the button 23 is depressed, the force is transferred to blister unit 26, and the pressure causes a frangible seal in the blister to rupture, releasing the test fluid through passage 27 into the lower section 22.

The blister unit 26 may be constructed and arranged in any suitable way. Such a device is used in the commercially available Elion system, https://atomodiagnostics.com/elion-rdt-platform/. However, the blister pack for producing a test fluid in the test system may be formed in the same way. Further explanation of such integrated fluid devices is provided in U.S. patent Ser. No. 10/595,763 and WO2018085878, the disclosures of which are hereby incorporated by reference.

While the blister type unit is illustrated, the present invention could be implemented with any suitable dispensing mechanism for the test fluid. It is preferred that the dispensing mechanism be separately formed and then fitted to the pre-mix vessel.

While this implementation shows two generally similar upper and lower parts, it will be understood that variations with different sized parts, different ways of opening and sealing, and different geometries for the test fluid release and reservoir are matters of design choice, and fall within the scope of this disclosure.

In use, the person being tested opens the pre-mix device 20, and produces saliva, for example by drooling into the vessel. Generally, a relatively small volume of saliva is required, for example between 60 μl and 500 μl. Markings may be provided inside the lower section 22 to indicate a minimum required level. Once the saliva is added, the user closes the device by engaging the upper section 20 with the lower section 22.

Once the device is closed, the button 23 is depressed, which compresses the blister pack 26, and as described releases the contained test fluid through passage 27 into the vessel, where it mixes with the saliva sample. In one form, the medical fluid may enter as a relatively high velocity stream, to enhance mixing with the saliva. The closed device may then be agitated (if required). Thus, the closed vessel contains a mixture of saliva and the test fluid. For certain tests, it may be required for this mixture to rest for some period before testing proceeds.

In an alternative implementation, the act of closing the device could directly trigger the release of the test fluid into the vessel.

FIG. 3 illustrates the engagement of the vessel 20 with the test unit 10. Vessel is placed onto the test unit, aligned with the shaped positioning features 15, and the slightly raised edge of sample port 11 engages the discharge port 28 of vessel 20. The sample port 11 ruptures the seal on the discharge port 28, which will then allow the mixture of saliva and test fluid to discharge into the sample port 11, and thereby onto the test material.

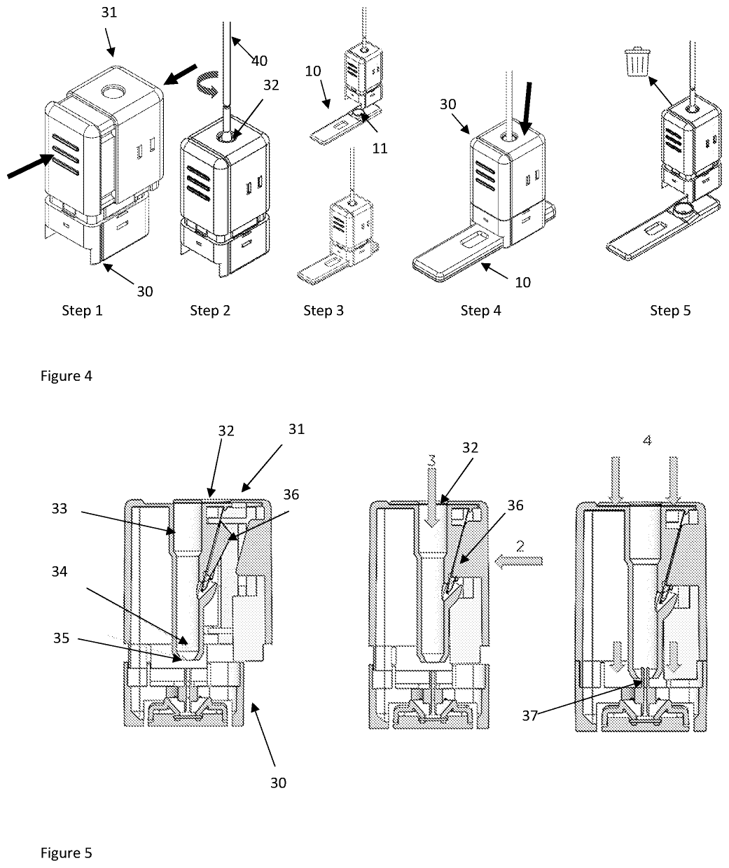

FIGS. 4 and 5 illustrate an alternative implementation of the present invention. Referring to FIG. 4, the stages in operation for the device commence with the left-hand figure, in which the premix unit 30 is in its resting condition. The shell 31 is movable relative to the unit 30 and is depressed to ready the unit for use. This performs two functions (as will be explained in more detail below): the hole 32 is aligned to the swab receptacle 33, and the test fluid is released into the swab receptacle 33.

At step 2, a swab 40 (which has previously been used as directed in order to gather, for example, a nasal sample) is inserted into the swab receptacle 33, and rotated in order to promote mixing. In this way, part of the sample from the swab 40 is discharged into the swab receptacle 33.

It will be appreciated that the swab receptacle, or vessel, is shaped and arranged so as to closely conform to, and ideally compress at least the outer surface, of the swab. As will be explained below, when the swab is then rotated to dislodge the sample from the swab into the test fluid, the interaction with the sides of the swab receptacle, and with any projections or ribs therein, will increase the effectiveness of sample extraction. The arrangement of the swab, being inserted into a relatively stable and fixed device, allows for effective and generally repeatable extraction processes, in comparison to arrangements where a vessel is squeezed manually, with variable user force, and hence highly variable extraction efficiency.

At step 3, after a suitable waiting period, dependent upon the specifics of the test (for example 1 minute), the pre-mix unit is aligned with the test cassette 10, and in particular, aligned so that the sample port 11 is aligned under the outlet valve of the pre-mix vessel. The swab may be left in, or in an alternative implementation, a snap off type swab may be used.

At step 4, the pre-mix vessel 30 is pressed down onto the test cassette 10. This will cause the release of the mixed sample and test fluid into the sample port 11 of the test cassette 10. In this way, the need for manual handling of the sample by the operator, for example by dripping from a dropper into the sample port, is avoided. Moreover, because the volume of fluid in the pre-mix vessel is not subject to control by the operator, and nor is the volume delivered from the outlet to the sample port, significant sources of test variation are removed.

At step 5, the pre-mix unit 30 is removed and disposed of. In an alternative implementation, the test unit could remain locked to the test cassette for disposal together. The latter, however, may not be suitable if the cassette is then intended for operation with an optical reader device.

FIG. 5 shows the internal mechanisms which achieve the steps of FIG. 4, according to one implementation. It will be appreciated that there are many similar or equivalent mechanical systems which could be used to achieve the same outcomes.

The left view shows the pre-mix unit 30 in the resting state. The moveable shell 31 is displaced to the right, the sample receptacle 33 is covered by part of the shell, and the blister unit 36 has not been activated. In this implementation, there is additionally a lyophilised buffer 34 in the lower section of the swab receptacle 33. The bottom of the sample receptacle is sealed with a foil layer 35.

In the central view, the shell 31 has been pressed inwards, which compresses the blister unit 36 and releases the test fluid into the sample receptacle 33. The shell 31 movement also places the opening 32 over the top of the sample receptacle, so that it is accessible for insertion of a swab, as explained above. The sample receptacle may include features, for example grooves or other structures to encourage turbulence, to assist in mixing. The sample may alternatively be acquired in another way, for example by drooling into the sample opening in suitable implementations.

In the right hand view, once the sample is collected and it is ready for delivery (as explained above), the pre-mix unit 30 is placed onto the test cassette 10, and pressed down to engage with the sample port 11. The act of pressing down causes the projecting tube 37 to pierce the foil and release the admixed sample, fluid and buffer into the sample port 11.

In the foil type delivery port, the port is initially sealed with pierceable layer of aluminium or polymer foil. Upon pressing down on the unit, a tube 37 with a sharp U shaped groove is designed to pierce into the foil and drain the premix fluid through the groove onto the cassette by the help of capillary force.

FIG. 9 illustrates an implementation of the present invention to a test unit which does not have any projecting features, and alignment is sufficiently achieved by the insertion of the test unit within the lower section of the pre-mix unit.

It will be understood that an advantage of the geometry of this implementation is that the swab is inserted from the top of the device into the vessel, the sample is extracted and mixed with the test fluid, and the mixed test fluid and sample are delivered from the bottom of the vessel into the sample port of the test unit. This vertical geometry means that there is no need to invert or handle the mixed sample, and so it cannot be inadvertently lost in that process.

In an alternative, a plug type design could be applied, in which the delivery port is sealed with a plug with soft material edges (e.g. rubber, flexible polymer) to ensure a good seal. Upon pressing the unit onto the cassette, the plug is lifted from its position and the fluid is drained through the grooves on the edge of the plug.

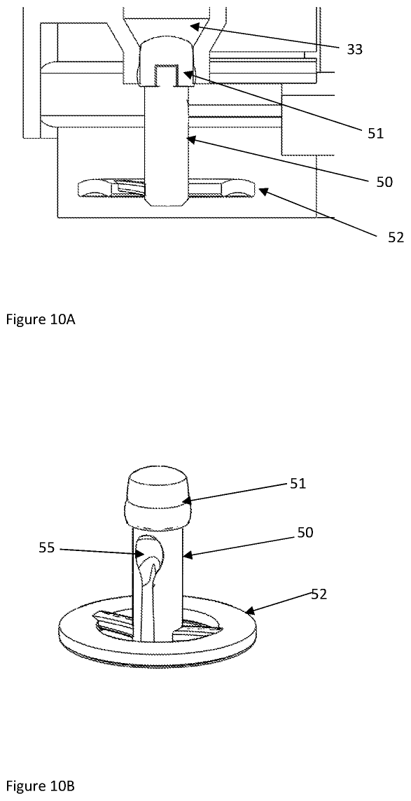

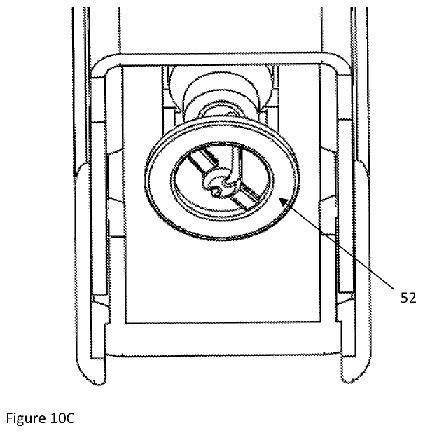

FIGS. 10A to 10C illustrates an implementation of a plug type valve design, a post 50 includes a plug 51, formed from a material such as a soft, solid polymer. The post 50 has a base 52 which engages the test unit. The post includes a groove or recess feature to guide the flow of mixed sample and pre-mix fluid into the sample port of the test unit. When the test unit us pressed down, post 50 interacts with the opening of the swab receptacle 33 to displace or break the plug, so that that fluid can flow from the receptacle 33 into the sample port.

The design of the base of the post can be modified to suit the sample port of any type of cassette. In this case, the base is circular, and it will ensure that the plug will only actuate when the sample port is properly aligned with the post and hence receptacle. The groove or similar features may of course be provided in many different shapes, and the post could be operated without such features.

Alternative release or valve type structures are possible, for example, the base of the receptable could be covered in a thinner layer of polymer, the piercing mechanism could be one of many alternative shapes, or a different valve mechanism could be employed.

It is preferred that the interlocking structures between the cassette and the base of the pre-mix unit are such that unless they are properly engaged, it is not possible to press down upon the unit and release the fluid.

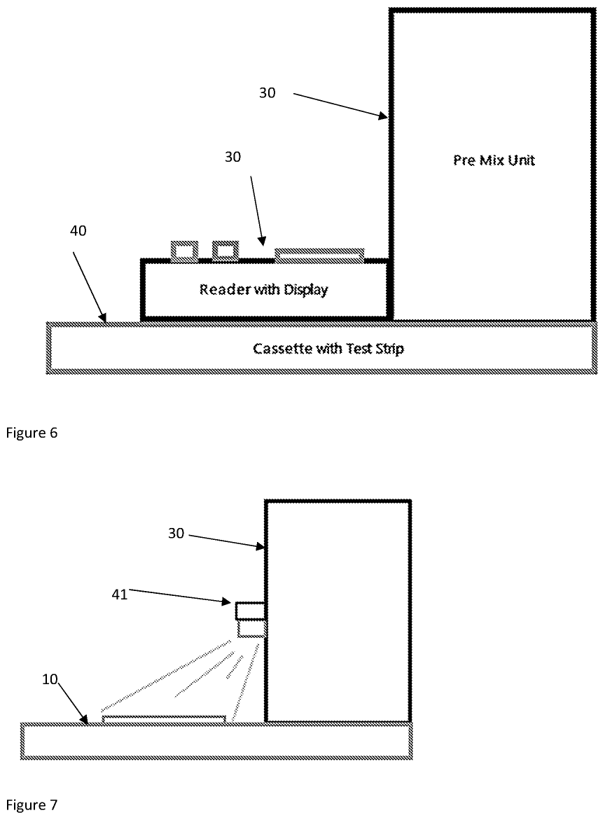

FIG. 6 illustrates an arrangement in which an additional reader unit 30 is provided, either as part of the cassette, or as a separate connectable unit. Such readers are known for, for example, pregnancy tests and similar applications, and in one form illuminate the test and control lines and process a received image. The reader unit could alternatively be formed as part of the pre-mix unit. In the latter case, the reader may only be made operative when the device is operated to release the sample and fluid, for example by pressing down as in FIG. 5.

FIG. 7 illustrates an alternative implementation in which a UV light (for example an LED) is position to illuminate the result window of the test cassette, for use in tests (for example certain COVID-19 tests) which require UV illumination. This may also incorporate a reader unit.

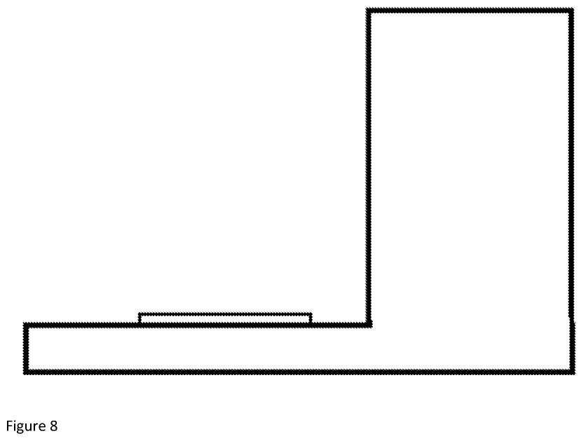

FIG. 8 illustrates an alternative integrated implementation, in which the pre-mix unit is integrated with the test cassette and the reader unit.

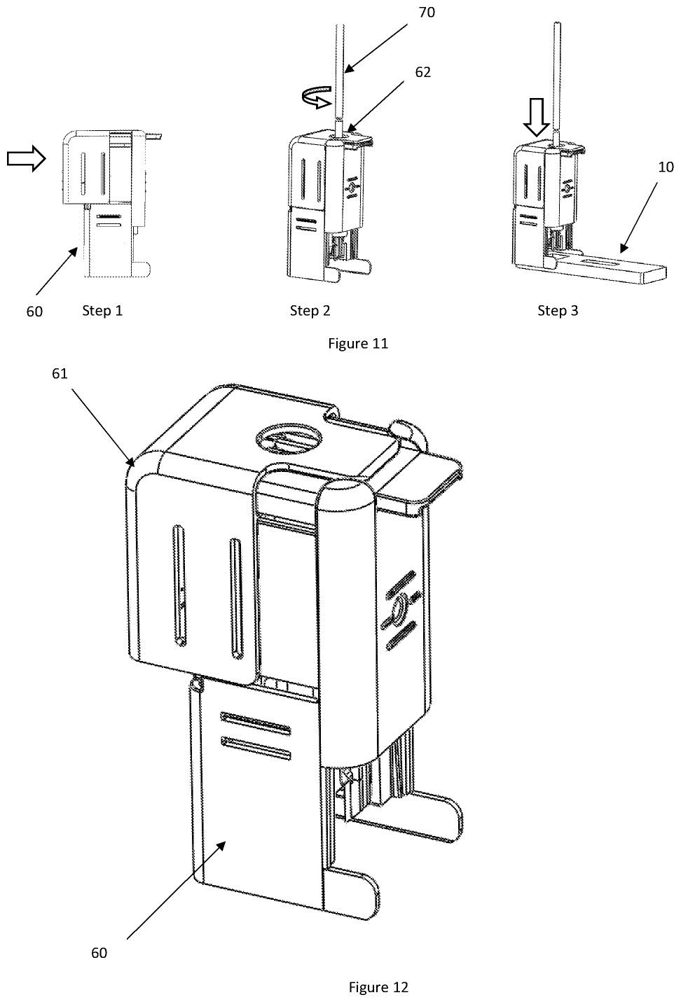

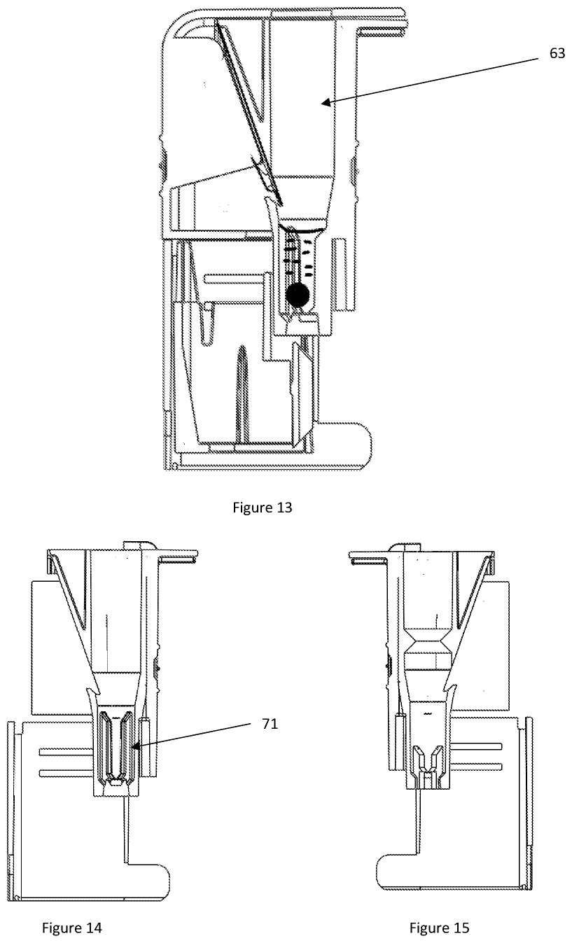

FIGS. 11 to 19 illustrate an alternative implementation of the present invention. FIG. 11 shows the stages of use of another alternative form of FIG. 4. Referring to FIG. 12, the premix unit 60 is in its resting condition. The shell 61 is movable relative to the unit 60 and is depressed to ready the unit for use. This performs two functions as shown in FIG. 13: the hole 62 is aligned to the swab receptacle 63, and the test fluid is released into the swab receptacle 63.

At step 2, a swab 70 is inserted into the swab receptacle 63, and rotated 3 to 4 times in order to promote mixing as shown in FIG. 16. In this way, part of the sample from the swab 70 is discharged into the swab receptacle 63. To ensure excellent sample transfer from the swab to the test fluid, ribs 71 may be employed in the receptacle 63. The ribs 71 also squeeze the swab 70 and prevent it from absorbing additional fluid. The ribs 71 may be vertical serrations as shown in FIG. 14 or funnel shaped as shown in FIG. 15. Of course, any suitable internal projections could be used.

Alternatively, a user may directly add drops of blood sample in the pre-mix unit 60 as shown in FIG. 19.

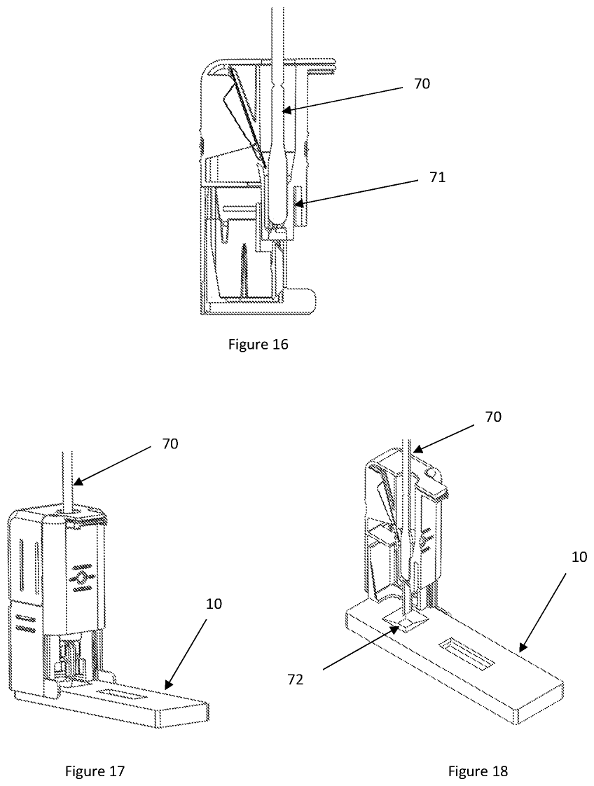

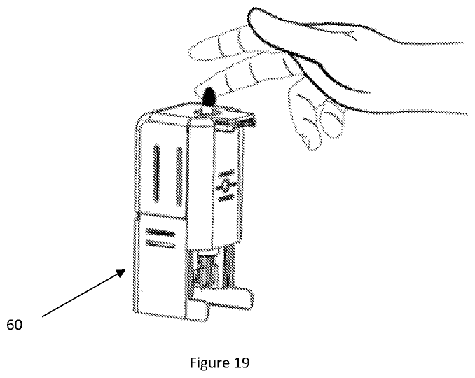

At step 3, after a suitable waiting period, dependent upon the specifics of the test (for example 1 minute), the pre-mix unit 60 is placed correctly on top of a test cassette 10 as shown in FIG. 17. Unlike the pre-mix unit in FIG. 4, users cannot deliver fluid without the test cassette 10 and the pre-mix unit 60 cannot be actuated unless it is placed on top of the test cassette 10. The swab may be left in, or in an alternative implementation, a snap off type swab may be used. The pre-mix vessel 60 is pressed down onto the test cassette 10. As shown in FIG. 18, this will cause the release of the mixed sample and test fluid into the sample port 72 of the test cassette 10.

The opening and release of fluid into the sample receptacle are in this implementation accomplished by part of the shell sliding relative to the rest of the body. It will be appreciated that there are many other comparable mechanisms which could be used to achieve this, for example by sliding, depressing a lever or button, rotation, opening or removing a frangible component, or any other suitable mechanism.

The term test fluid is intended to be broadly understood. The invention is not limited to any specific kind of fluid being contained—the fluid will be whatever is required by the test protocol to be premixed for a specific test. It may be a buffer solution, reagent, or any other required liquid. When the swab is inserted into the sample port, the absorbent material is compressed, and the sample is released into the sample port. The sample port is positioned directly above the test strip's sample pad. User then presses blister button to release the test fluid from the reservoir into the sample port. The two fluids mix and are transferred across the test strip by capillary action.

It will be appreciated that implementations of the present invention allow for the fluid volume to be controlled, so that allow sufficient fluid is released from the blister unit (or other release mechanism) for the purposes of the test, with allowance for losses during transfer, but no more. In principle, implementations of the present invention allow for substantially all of the sample which has dispersed into the test fluid to be presented to the test unit. As a result, this approach allows for an increased concentration of the sample within the test fluid (and hence an increased concentration of the component(s) to which the test is sensitive) to be delivered.

In some cases, it may be possible to have an additional dry reagent such as a lyophilized chemical in the vessel. In those cases, the vessel will facilitate mixing of sample, test fluid and the dry reagent.

It will be understood that the shaped features on the test unit, both for positioning and for engaging the discharge port, will be complementary to corresponding features on the base of the vessel. However, as described above the present invention may be implemented without any special features on the test unit.

Although the vessel and test unit have been described as separate components, the present invention may be implemented with the vessel integrally formed with the test unit.

Further, although the invention has been described with the test fluid release being a separate action after the vessel is sealed, in alternative implementations the release may be triggered once the vessel is closed or sealed, or when the vessel is engaged with the test unit.

Variations and additions are possible within the general scope of the invention disclosed, as will be apparent to those skilled in the art.

1. A pre-mix device for use with a test unit, the device comprising: a vessel, a sample opening and a discharge port, the discharge port being separate from the sample opening, wherein the vessel is operatively adapted to receive a sample via the sample opening, so that when the pre-mix device is operatively engaged with the test unit, the contents of the vessel are released from the discharge port to the test unit.

2. A pre-mix device according to claim 1, wherein the pre-mix device further includes an integrated reservoir of test fluid, and the reservoir is adapted to discharge test fluid from the reservoir into the vessel, so that the vessel operatively discharges a mixture of test fluid and sample to the test unit.

3. A pre-mix device according to claim 1, wherein the discharge port is sealed and selectively openable by engagement of the pre-mix device with the test unit.

4. A pre-mix device according to claim 2, wherein the opening is closed until a movement of a section of the body of the pre-mix device.

5. A pre-mix device according to claim 4, wherein the the test fluid is released by the same movement as causes the sample opening to be operable.

6. A pre-mix device according to claim 2, wherein the volume of test fluid is selected to deliver substantially the test volume required for the test unit, so that operatively, when the substantially all the mixed sample and test fluid is adapted to be discharged to the test unit via the discharge port.

7. A pre-mix device according to sample opening is at the top of the vessel, and the discharge port is at the base of the vessel.

8. A pre-mix device according to claim 1, wherein the sample opening is adapted to allow a swab carrying a sample to be inserted, the shape and dimensions of the vessel being such that swab is closely engaged with internal walls of the vessel, so as to assist in removal of the sample from the swab.

9. A pre-mix device according to claim 2, wherein the pre-mix device further includes an interlock, so that unless a test unit is engaged with the base of the device, the mixture of the sample and the test fluid cannot be discharged through the discharge port.

10. A pre-mix device according to claim 1, wherein the vessel further includes a dry reagent.

11. A pre-mix device according to claim 10, wherein the dry reagent is lyophilised buffer.

12. (canceled)

13. (canceled)

14. (canceled)

15. A medical test device, comprising:
a test unit including a body, a test material and a sample port to access the test material; and a pre-mix device according to any one of the preceding claims,
such that in use a sample is received into the pre-mix device, mixed with a test fluid, and when the pre-mix device is operatively engaged with the test unit, a mixture of the sample and the test fluid is discharged into the sample port, and thereby onto the test material.

16. A medical test device according to claim 11, wherein the test unit and the pre-mix device are formed separately, and the test unit and/or the pre-mix device include surface features to assist in alignment of the device with the sample port of test unit.

17. (canceled)

18. A medical device according to claim 13, wherein the discharge port is opened during engagement of the base pre-mix unit with the test unit.

19. A medical device according to claim 13 wherein the medical device includes an integrated light source, positioned to illuminate a test result window of the test unit.

20. A medical device according to claim 11, further including an integrated reader to determine the test outcome and a display to show the determined outcome.
